(19) United States
(12) Patent Application Publication
(10) Pub. No.: US 2024/0032905 A1
(43) Pub. Date: Feb. 1, 2024

(54) DEVICES AND METHODS FOR THE TREATMENT OF HEART FAILURE

(71) Applicant: Corvia Medical, Inc., Tewksbury, MA (US)

(72) Inventor: David Stephen CELERMAJER, Sydney (AU)

(21) Appl. No.: 18/321,670

(22) Filed: May 22, 2023

Related U.S. Application Data
(63) Continuation of application No. 16/854,100, filed on Apr. 21, 2020, now Pat. No. 11,690,609.
Application No. 16/854,100 is a continuation of application No. 12/447,617, filed on Apr. 28, 2009, now Pat. No. 10,624,621.

(30) Foreign Application Priority Data
Nov. 7, 2006 (AU) 2006906202

(51) Int. Cl. A61B 17/02; A61B 17/00; A61F 2/06; A61F 2/24; A61M 27/00

(52) U.S. Cl. CPC A61B 17/02; A61B 17/0057; A61F 2/06; A61F 2/2412; A61F 2/2475; A61F 2/24; A61F 2/2442; A61M 27/002; A61B 2017/00252; A61B 2017/00575; A61B 2017/00592; A61B 2017/00606; A61B 2017/00867; A61B 2017/0237

(57) ABSTRACT

A device for treating heart failure in a patient. The device comprising a body, at least one passage through the body, at least one one way valve in the passage and a mounting means adapted for mounting the body in an opening provided in the patient's atrial septum. In use, the device is oriented such that, when the patient's left atrial pressure exceeds the patient's right atrial pressure by a predetermined amount, the one way valve(s) opens to allow blood flow through the passage(s) from the left atrium to the right atrium to thereby reduce the left atrial pressure.

### CROSS REFERENCE TO RELATED APPLICATIONS

This application is a continuation of U.S. application Ser. No. 16/854,100, filed Apr. 21, 2020, which is a continuation of U.S. application Ser. No. 12/447,617, filed Apr. 28, 2009, now U.S. Pat. No. 10,624,621 which is a 371 of International Application No. PCT/AU2007/001704, filed Nov. 7, 2007, which application claims priority to Australian Application No. 2006906202, filed Nov. 7, 2006, each of which is herein incorporated by reference in its entirety for all purposes.

### FIELD OF THE INVENTION

The present invention relates generally to devices and methods for the treatment of heart failure and, more particularly, to devices and methods for the relief of high pressure in the cardiovascular system to alleviate symptoms of cardiovascular disease.

### BACKGROUND OF THE INVENTION

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterised by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. In addition to diastolic heart failure, a number of other medical conditions, including systolic dysfunction of the left ventricle and valve disease, can lead to elevated pressures in the left atrium. Increased left atrial pressure often causes acute or chronic breathlessness amongst other problems. In addition, a variety of heart conditions can lead to “right heart failure”, which can result in enlargement of the liver (hepatomegaly), fluid accumulation in the abdomen (ascites) and/or swelling of the lower limbs.

In the past, strategies have been described for the relief of high pressure in the right atrium, such as the creation of hole(s) in the native or surgically created septum between the left and right atria. These have been designed for the rare conditions of pulmonary hypertension or cavopulmonary connections for certain complex congenital heart diseases. O'Loughlin et al. recently described a fenestrated atrial septal defect closure device for the palliation of advanced pulmonary hypertension. However, this device allows bidirectional flow, and the passage of thrombi, and was shown to be closed over within 6 months of insertion. Thus a need still exists for devices to relieve high pressure in the left atrium and which will prevent or minimize the chance of the passage of thrombi.

Accordingly, there exists a need for devices and methods to treat heart failure particularly diastolic and/or systolic failure of the left ventricle and its consequences.

### SUMMARY OF THE INVENTION

According to a first aspect of the invention, there is provided a device for treating heart failure in a patient, the device comprising:
a body;
at least one passage through the body;
at least one one way valve in the passage; and
a mounting means adapted for mounting the body in an opening provided in the patient's atrial septum,

wherein, in use, the device is oriented such that, when the patient's left atrial pressure exceeds the patient's right atrial pressure by a predetermined amount, the one way valve(s) opens to allow blood flow through the passage(s) from the left atrium to the right atrium to thereby reduce the left atrial pressure.

According to a second aspect of the invention, there is provided a device for treating heart failure or pulmonary venous hypertension in a patient, the device comprising:
a body;
at least one passage through the body;
at least one one way valve in the passage; and
a mounting means adapted for mounting the body in the patient's venous system,

wherein, in use, the device is oriented such that the one way valve(s) prevents blood flow through the passage(s) in a direction opposite to that of the natural flow direction.

The device is preferably adapted to be fitted into a blood vessel in the patient's venous system, such as the inferior vena cava, superior vena cava, the hepatic vein, an iliac vein, or one or more pulmonary veins.

According to a third aspect of the invention, there is provided a device for treating lower limb venous hypertension in a patient, the device comprising:
a body;
at least one passage through the body;
at least one one way valve in the passage; and
a mounting means adapted for mounting the body in the patient's lower limb venous system,

wherein, in use, the device is oriented such that the one way valve(s) prevents blood flow through the passage(s) in a direction opposite to that of the natural flow direction.

The above device is also suitable for treating varicose veins.

The body is preferably in the form of a stent, most preferably an expandable stent.

The valve is preferably a duckbill valve, a leaflet valve, a flap valve, a disc in cage type valve or a ball in cage type valve. The valve is preferably biased to a closed position, most preferably by the inherent resilience of the valve material. The valve preferably opens when the predetermined amount of pressure differential is at least approximately 2 mm Hg, preferably approximately 5 to 25 mm Hg, even more preferably 5 to 15 mm Hg.

In one form, the device has a single passage through the body, most preferably centrally located in relation to the body. In another form, the device has a single passage through the body, most preferably eccentrically located in relation to the body. In yet another form, the device has a plurality of passages through the body, each with a one way valve therein, most preferably each eccentrically located in relation to centre of the body.

According to a fourth aspect of the invention, there is provided a device for treating heart failure in a patient, the device comprising:
a body;
at least one passage through the body;
a mesh or grill arrangement within the passage and having apertures therein of a size permitting flow of blood, whilst substantially excluding thrombi, therethrough;
a mounting means adapted for mounting the body in an opening provided in the patient's atrial septum,

wherein, in use, the device allows blood flow through the passage(s) from the left atrium to the right atrium when the patient's left atrial pressure exceeds the patient's right atrial pressure to thereby reduce the patient's left atrial pressure.

The device preferably includes a mesh or grill arrangement across one or both ends of the passage(s).

The apertures preferably have a maximum dimension of less than 4 mm, most preferably less than 2 mm. The mesh or grill is preferably coated or impregnated with one or more drugs, adapted for preventing thrombosis or endothelialisation of the opening in the patient's atrial septum, including an anticoagulant substance, such as heparin, or an inhibitor of re-endothelialisation, such as sirolimus or paclitaxel.

In one form, the device has a single passage through the body, most preferably centrally located in relation to the body. In another form, the device has a plurality of passages through the body, each with a mesh or grill arrangement therein, most preferably each eccentrically located in relation to centre of the body.

The device is preferably flexible, most preferably formed from a material which can be deformed but later return to its original shape. An example of such a material is Nitinol.

The device is preferably collapsible and adapted for implanting via a catheter, although it could be inserted at surgery.

The device is preferably collapsible to a size able to pass through an opening made in the patient's atrial septum (or an enlargement of a pre-existing communication, by standard methods) and adapted to return to a shape where at least some of the device would have been unable to pass through the opening in the patient's atrial septum. The device is preferably formed from a Nitinol mesh, or any other material which can be deformed but later return to its original shape.

The mounting means preferably comprises at least one flange having a dimension larger than the opening in the patient's septum. More preferably, the mounting means preferably comprises a pair of spaced apart flanges having a dimension larger than the opening in the patient's septum.

The external dimension of the body, remote the flange(s), is preferably substantially equal to the opening in the patient's atrial septum.

In one embodiment, the flanges are adapted for gluing, suturing, stapling or pinning to the patient's septum.

In another embodiment, the flanges are spaced apart by about the thickness of the patient's atrial septum and are adapted to locate, most preferably by gripping, the patient's atrial septum therebetween.

According to a fifth aspect of the invention, there is provided a method for treating heart failure in a patient, the method comprising the steps of:
forming an opening in the patient's atrial septum;
inserting at least one one way valve in the opening that is oriented such that the one way valve(s) allows blood flow through the passage from the left atrium to the right atrium when the patient's left atrial pressure exceeds the patient's right atrial pressure; and
securing the one way valve(s) relative to the patient's atrial septum,

whereby, when the patient's left atrial pressure exceeds the patient's right atrial pressure by a predetermined amount, the valve opens to allow blood flow through the passage(s) from the left atrium to the right atrium to thereby reduce the patient's left atrial pressure.

The above method is particularly suited for treating cardiovascular disease manifest by left atrial hypertension, such as that due to left ventricular systolic or diastolic dysfunction.

The predetermined amount of pressure differential is at preferably least approximately 3 mm Hg, preferably approximately 5 to 25 mm Hg, even more preferably 5 to 15 mm Hg.

According to a sixth aspect of the invention, there is provided a method for treating heart failure in a patient, the method comprising the steps of:
forming an opening in the patient's atrial septum;
inserting a mesh or grill arrangement within the opening having apertures therein of a size permitting passage of blood, whilst substantially excluding passage of thrombi, therethrough; and
securing the mesh or grill arrangement relative to the patient's atrial septum.

The mesh or grill arrangement is preferably provided within a passage in a body, and the method preferably includes the step of securing the body relative to the patient's atrial septum.

The above method is particularly suited for treating cardiovascular disease manifest by left atrial hypertension, such as that due to left ventricular systolic or diastolic dysfunction.

According to a seventh aspect of the invention, there is provided a method for treating heart failure in a patient, the method comprising the steps of:
inserting at least one one way valve in the patient's venous system that is oriented such that the one way valve(s) prevents blood flow through the said venous system in a direction opposite to that of the natural flow direction; and
securing the one way valve(s) relative to the patient's venous system.

The method preferably includes the steps of inserting and securing the one way valve in the patient's blood vessel, such as the inferior vena cava, superior vena cava, the hepatic vein, an iliac vein, or one or more pulmonary veins.

The method preferably includes a step of inserting and securing, most preferably by expanding, a stent with the one way valve(s) therein.

According to an eighth aspect of the invention, there is provided a device for treating heart failure in a patient, the device comprising:
a tube having first and second ends in fluid communication with the left and right atriums of the heart respectively; and
a valve between the first and second ends and adapted to selectively prevent or allow fluid flow through the tube,

wherein, in use, when the patient's left atrial pressure exceeds the patient's right atrial pressure by a predetermined amount, the valve opens to allow blood flow through the tube from the left atrium to the right atrium to thereby reduce the left atrial pressure.

The valve opens when the predetermined amount of pressure differential is at preferably least approximately 2 mm Hg, preferably approximately 5 to 25 mm Hg, even more preferably approximately 5 to 15 mm Hg.

According to an ninth aspect of the invention, there is provided a device for treating heart failure or pulmonary venous hypertension in a patient, the device comprising:
a tube having first and second ends in fluid communication with the left and right atriums of the heart respectively; and
a one way valve in the tube,

wherein, in use, the one way valve prevents blood flow through the tube from the right atrium to the left atrium.

According to a tenth aspect of the invention, there is provided a method for treating heart failure in a patient, the method comprising the steps of:
connecting a tube externally between the patient's left and right atriums; and
inserting a one way valve in the tube that is oriented such that the one way valve allows blood flow through the passage from the left atrium to the right atrium when the patient's left atrial pressure exceeds the patient's right atrial pressure,

whereby, when the patient's left atrial pressure exceeds the patient's right atrial pressure, by a predetermined amount, the valve open to allow blood flow through the passage(s) from the left atrium to the right atrium to thereby reduce the patient's left atrial pressure.

The predetermined amount of pressure differential is at preferably least approximately 2 mm Hg, preferably approximately 5 to 25 mm Hg, even more preferably approximately 5 to 15 mm Hg.

### BRIEF DESCRIPTION OF THE DRAWINGS

Preferred embodiments of the invention will now be described, by way of examples only, with reference to the accompanying drawings in which:

FIG. 1 is a front view of a first embodiment of a device for treating heart failure;

FIG. 2 is a cross sectional side view of the device shown in FIG. 1;

FIG. 3 is a cross sectional side view of the device shown in FIGS. 1 and 2 implanted in a human heart;

FIG. 4 is a rear view of the device shown in FIG. 1;

FIG. 5 is a front view of a second embodiment of a device for treating heart failure;

FIG. 6 is a front view of a third embodiment of a device to treat heart failure;

FIG. 7 is a cross sectional side view of the device shown in FIG. 6;

FIG. 8 is a front view of a fourth embodiment of a device to treat heart failure;

FIG. 9 is a cross sectional side view of the device shown in FIG. 8;

FIG. 10 is a cross sectional side view of a fifth embodiment of a device for treating heart failure;

FIG. 11 is a cross sectional side view of the device shown in FIG. 10 implanted in a patient's inferior vena cava;

FIG. 12 is a cross sectional side view of a first embodiment of a delivery mechanism for the device shown in FIG. 10;

FIG. 13 is a cross sectional side view of a second embodiment of a delivery mechanism for the device shown in FIG. 10;

FIG. 14 is a cross sectional side view of a sixth embodiment of a device for treating heart failure implanted in a patient's hepatic vein;

FIG. 15 is a cross sectional side view of a pair of the devices shown in FIG. 14 implanted in a patient's iliac veins;

FIG. 16 is a front view of a seventh embodiment of a device for treating heart failure;

FIG. 17 is a front view of an eighth embodiment of a device for treating heart failure;

FIG. 18 is a cross sectional side view of the device shown in FIG. 17;

FIG. 19a is a front view of a ninth embodiment of a device for treating heart failure;

FIG. 19b is a cross sectional side view the device shown in FIG. 19a;

FIG. 20 is a front view of a tenth embodiment of a device for treating heart failure,

FIG. 21a is a cross sectional side view of an eleventh embodiment of a device for treating heart failure, collapsed within a catheter;

FIG. 21b is a cross sectional perspective view of the device shown in FIG. 21a, collapsed within a catheter;

FIG. 22a is a cross sectional side view of the device shown in FIG. 21a, partially deployed from the catheter;

FIG. 22b is a cross sectional perspective view of the device shown in FIG. 21a, partially deployed from the catheter;

FIG. 22c is an enlarged, partial cross sectional side view of the device shown in FIG. 21a, partially deployed from the catheter;

FIG. 23a is a side view of the device shown in FIG. 21a, deployed from the catheter;

FIG. 23b is a cross sectional side view of the device shown in FIG. 21a, deployed from the catheter; and

FIG. 24 is a cross sectional side view of a twelfth embodiment of a device for treating heart failure.

### DETAILED DESCRIPTION OF THE PREFERRED EMBODIMENTS

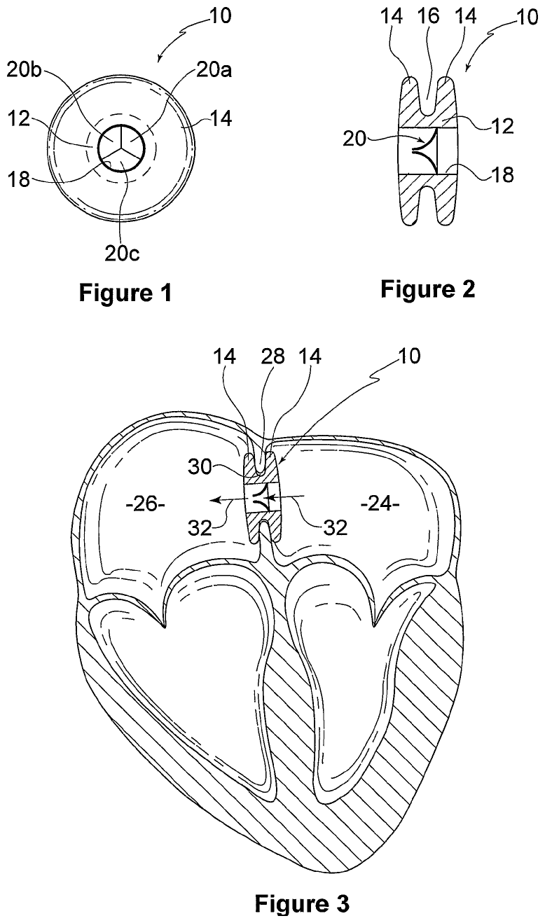

FIGS. 1 to 4 show a first embodiment of a device 10 for treating heart failure. The device 10 includes a generally cylindrical body 12 with a mounting means, in the form of a pair of annular flanges 14 at either end with an annular gap 16 therebetween. The body 12 has a centrally located passage or duct 18 within which is provided a one way valve 20, in the form of three flexible valve leaflets 20a to 20c.

The external diameters of the body 12, the flanges 14 and internal diameter of the passage 18 are approximately 18, 38 and 12 mm respectively. In other embodiments (not shown), the diameter of the body 12 ranges from 8 to 25 mm, the diameter of the flanges 14 ranges from 20 to 50 mm, and the diameter of the passage 18 ranges from 4 to 15 mm.

FIG. 3 shows a patient's heart 22 with a left atrium 24 and a right atrium 26 separated by an atrial septum 28. The device 10 is mounted within a generally circular opening 30 made in the septum 28 and with the edges of the septum 28 adjacent the opening 30 positioned in the gap 16 between the flanges 14. The opening 30 has an internal diameter approximately equal to the external diameter of the body 12. The device 10 is retained adjacent the opening 30 in the septum 28 as the flanges 14 are larger, and thus cannot pass through, the opening 30. Alternatively, or in addition, one or both of the flanges 14 can be glued, sutured, stapled or pinned to the patient's septum 28 to secure the device 10 thereto.

The device 10 can be implanted during open heart surgery or percutaneously using a catheter. In either case, the opening 30 is firstly fashioned in the patient's atrial septum 28. Some or all of the device 10 is then collapsed to a size able to pass through the opening 30 and subsequently expanded to the configuration shown in FIG. 3. Forming the s body 12 and the flanges 14 of the device 10 from a Nitinol wire mesh result in it being suitable for implanting in a manner similar to the implanting of the AMPLATZER (Trade Mark) septal occluder produced by AGA Medical Corp. More particularly, the exterior faces of the flanges 14 are pulled away from one another which causes the device 10 to lengthen and simultaneously reduce in diameter for fitting within a catheter able to pass through the opening 30. When the separating force is then removed the flanges 14 return to the (expanded) configuration in FIGS. 1 to 4.

The device 10 is orientated during implanting with the one way valve 20 only allowing blood flow through the passage 18 from the left atrium 24 to the right atrium 26, as indicated by arrows 32. More particularly, when the left atrial pressure exceeds the right atrial pressure by about 5-15 mm Hg, the valve leaflets 20a to c separate and thus open the passage 18 to blood flow from the left atrium 24 to the right atrium 26.

The leaflets 20a to 20c are formed from biological, mechanical or engineered tissue and are inherently biased towards a closed position. Further, the patient's right atrial pressure exceeding the left atrial pressure also assists in the closing, and the maintaining closed, of the valve 20.

The relief and/or avoidance of the left atrial pressure significantly exceeding the right atrial pressure is beneficial in alleviating the adverse consequences of left atrial hypertension complicating cardiovascular diseases, including left ventricular systolic and/or diastolic dysfunction and/or valvular diseases.

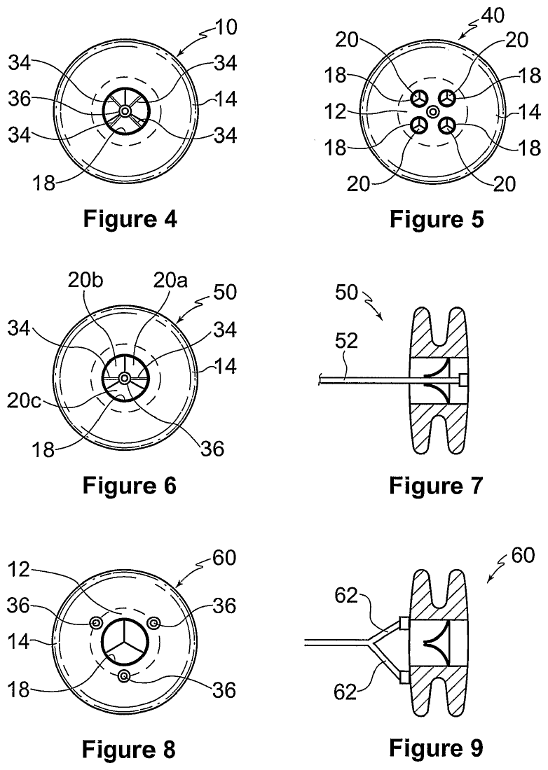

As best seen in FIG. 4, the device 10 includes four thin collapsible struts 34 connected to a central fixture or boss 36 having an internally threaded opening. A cable (not shown) is threadedly attachable to the fixture 36. The fixture 36 is accessible from the left atrium.

To implant the device 10, it is firstly collapsed inside a catheter. When the catheter is correctly positioned adjacent the opening 30, the cable is used to push the device 10 out of the catheter, whereafter it expands to the shape shown in FIG. 3. The cable is then unscrewed from the fixture 36 and removed from the patient with the catheter.

The device 10 can also be adapted to allow later removal by a percutaneous route, for example by the placement of small hooks (not shown) on a surface of the device 10 that is closest to a nearby venous access site.

FIGS. 5 shows a second embodiment of a device 40 for treating heart failure. The construction, function and implanting of the device 40 is similar to that of the device 10 and like reference numerals are used to indicate like features between the two embodiments. However, the device 40 has four eccentrically located passages 18 through the body 12 and blood flow therethrough is controlled by four corresponding sets of valve leaflets 20.

FIGS. 6 and 7 show a third embodiment of a device 50 for treating heart failure. The construction, operation and implantation of the device 50 is similar to that of the device 10 and like reference numerals are used to indicate like features between the two embodiments. However, the device 50 has only one collapsible strut 34 connected to a central fixture 36, to which a cable 52 can be attached. The fixture 36 is also accessible from the left atrium. In a variation of this embodiment, the fixture is accessible from the right atrium.

FIGS. 8 and 9 show a fourth embodiment of a device 60 for treating heart failure. The construction, function and implanting of the device 60 is similar to that of the device 10 and like reference numerals are used to indicate like features between the two embodiments. However, the device 60 has three fixtures 36 attached to the body 12, adjacent the passage 18, to which three respective cables 62 (see FIG. 9) can be attached. The fixtures 36 are accessible from the right atrium.

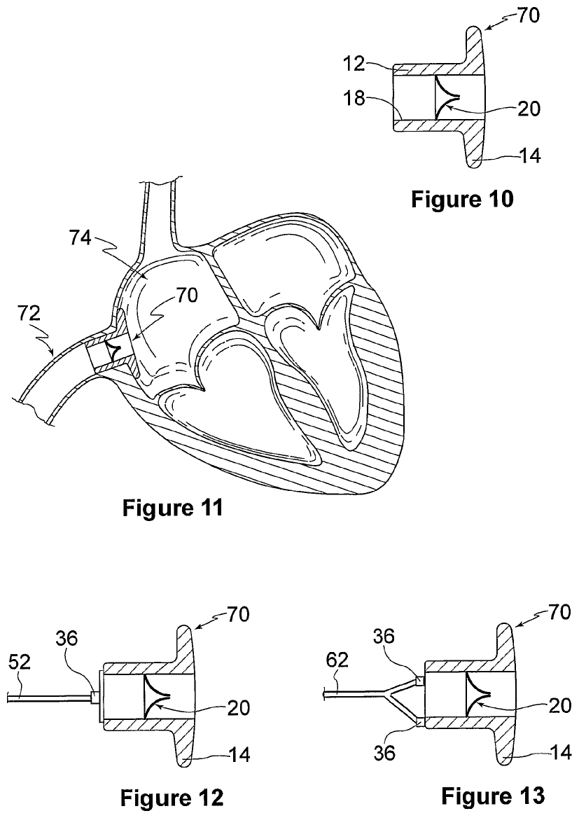

FIGS. 10 and 11 show a fifth embodiment of a device 70 for treating heart failure, in a manner similar to that of the device 10. However, unlike the earlier embodiments, the device 70 only has a single mounting flange 14 which, as shown in FIG. 11, makes it suitable for implanting in the inferior vena cava 72 at or near the junction with the right atrium 74. The device 70 is preferably produced from a deformable material that can resume its preformed shape (such as Nitinol) and may be implanted by a percutaneous approach.

More particularly, the device 70 is collapsed and introduced in the venous system within a sheath, and removed from the sheath to expand when correctly positioned.

FIGS. 12 and 13 show two mechanisms suitable for delivering the device 70 to the inferior vena cava. The mechanism shown in FIG. 12 is similar to that shown in FIGS. 6 and 7 and the mechanism shown in FIG. 13 is similar to that shown in FIGS. 8 and 9.

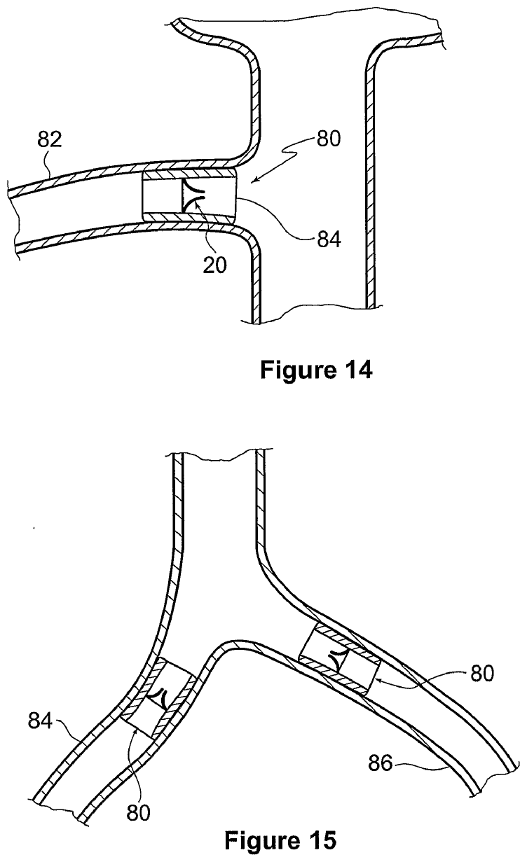

FIG. 14 is a cross sectional side view of a sixth embodiment of a device 80 for treating heart failure, implanted in a patient's hepatic vein 82. The device 80 does not include any mounting flanges and its body is instead an expandable stent 84 with a one way valve 20 therein.

FIG. 15 shows an alternative implanting of the device 80 in a patient's iliac veins 84 and 86.

The device 80 is also suitable for placement in the venous system of the lower limb or iliac system to relieve the signs or symptoms of lower limb hypertension (e.g., peripheral oedema and/or varicose veins).

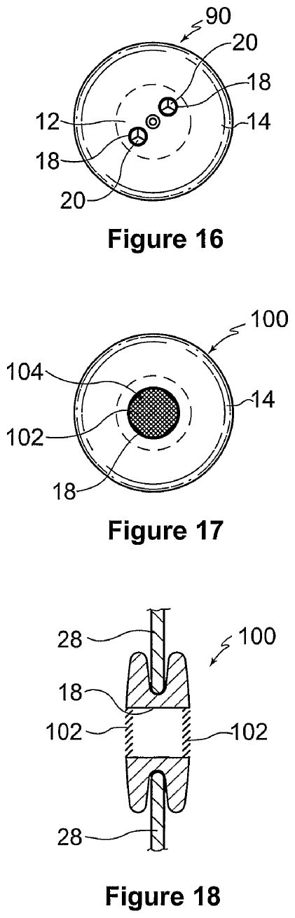

FIG. 16 shows a seventh embodiment of a device 90 for treating heart failure. The construction, function and implanting of the device 90 is similar to that of the device 40 and like reference numerals are used to indicate like features between the two embodiments. However, the device 90 has only two eccentrically located passages 18 through the body 12 and blood flow therethrough is controlled by two corresponding sets of valve leaflets 20.

FIG. 17 shows an eighth embodiment of a device 100 for treating heart failure. This embodiment is constructed and implanted in a similar manner to that previously described. However, the device 100 has a passage 18 therethrough with a mesh or grill arrangement 102 across each end of the passage 18. The mesh 102 has apertures 104 therein of a maximum dimension of less than 4 mm which permit the flow of blood from the left to the right atrium through the passage 18, whilst substantially excluding thrombi. The mesh 102 is coated or impregnated with one or more drugs, adapted for preventing thrombosis or endothelialisation of the opening in the patient's atrial septum, including an anticoagulant substance, such as heparin, or an inhibitor of re-endthelialisation, such as sirolimus or paclitaxel.

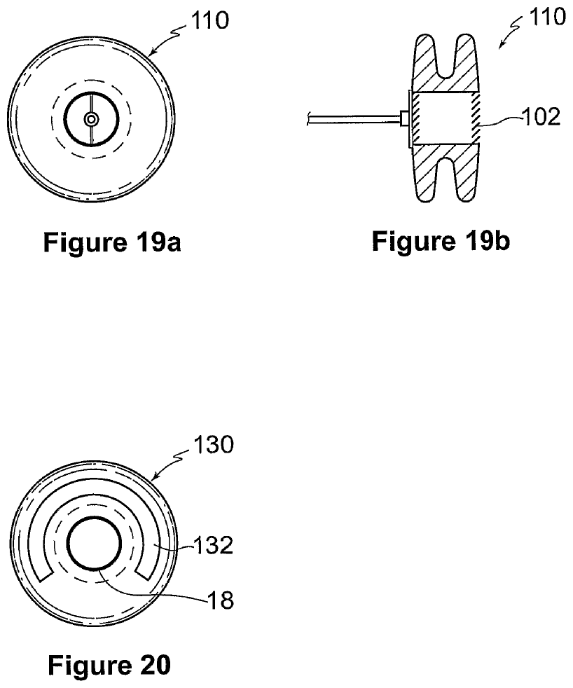

FIGS. 19a and 19b show a ninth embodiment of a device 110 for treating heart failure. The construction, operation and implantation of the device 110 is similar to that of the device 10 and like reference numerals are used to indicate like features between the two embodiments. The device 110 utilizes a strut/fixture arrangement similar to that shown in FIGS. 6 and 7.

FIG. 20 shows a tenth embodiment of a device 130 for treating heart failure. The construction, operation and implantation of the device 130 is similar to that of the device 10 and like reference numerals are used to indicate like features between the two embodiments. The device 130 has a helical groove 132 for releasably engaging a corresponding fitting on the end of a catheter cable during implantation.

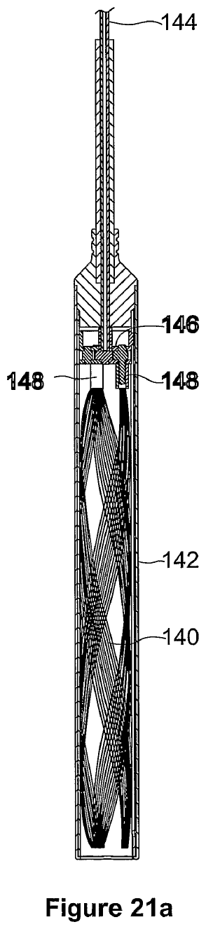

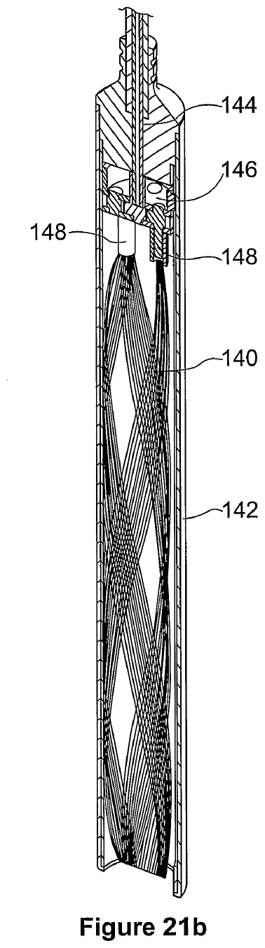

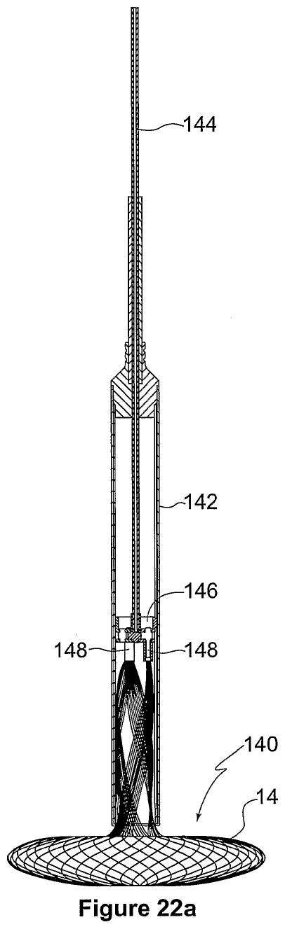

FIGS. 21a to 23b show an eleventh embodiment of a device 140 for treating heart failure. The construction, operation and implantation of the device 100 is similar to that of the device 10 and like reference numerals are used to indicate like features between the two embodiments. The body 12 and the flanges 14 of the device 140 are formed from a Nitinol wire mesh which result in it being suitable for implanting in a manner similar to the implanting of the AMPLATZER (Trade Mark) septal occluder produced by AGA Medical Corp. The device 140 is collapsed by pulling the exterior faces of the flanges 14 away from one another which causes the device 140 to lengthen and simultaneously reduce in diameter. When the separating force is removed the flanges 14 return to the (expanded) configuration.

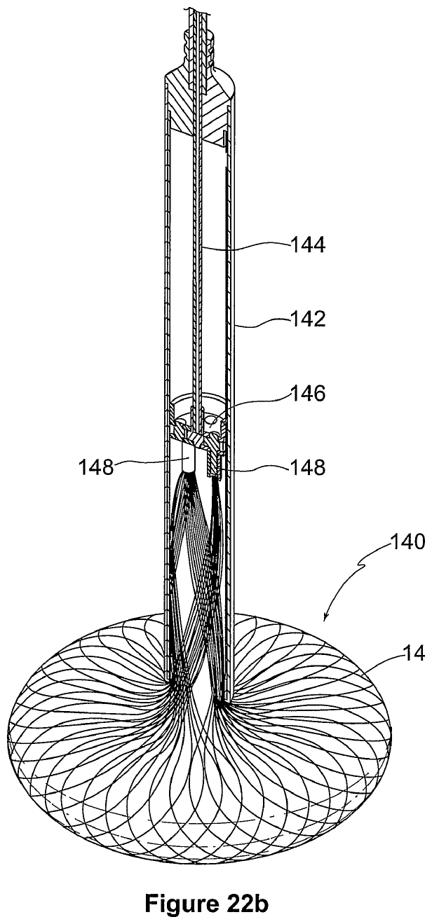

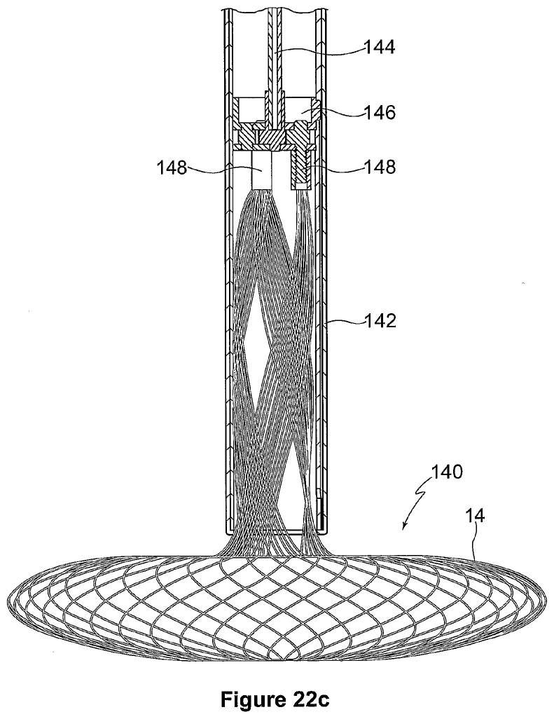

More particularly, as shown in FIGS. 21a and 21b, the device 140 is initially collapsed within a catheter 142 of about 5 mm in diameter, which is able to pass through an opening in the septum. As shown in FIGS. 22a to 22c, the device 140 is then partially deployed from the catheter 142 by movement of wire 144, and thus head 146, relative to the catheter 142. This results in part of the device 140 expanding to form the first flange 14.

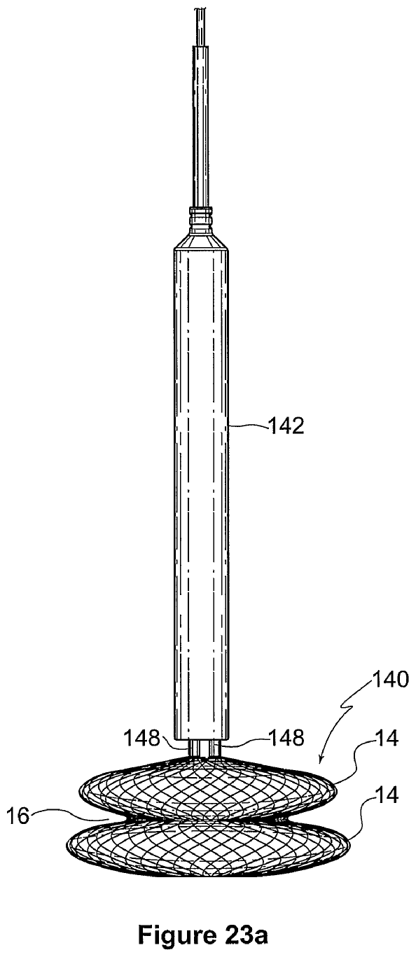

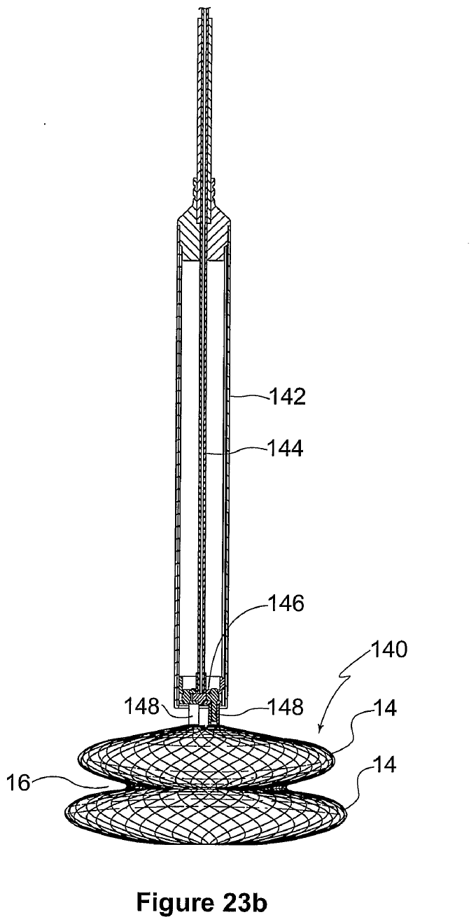

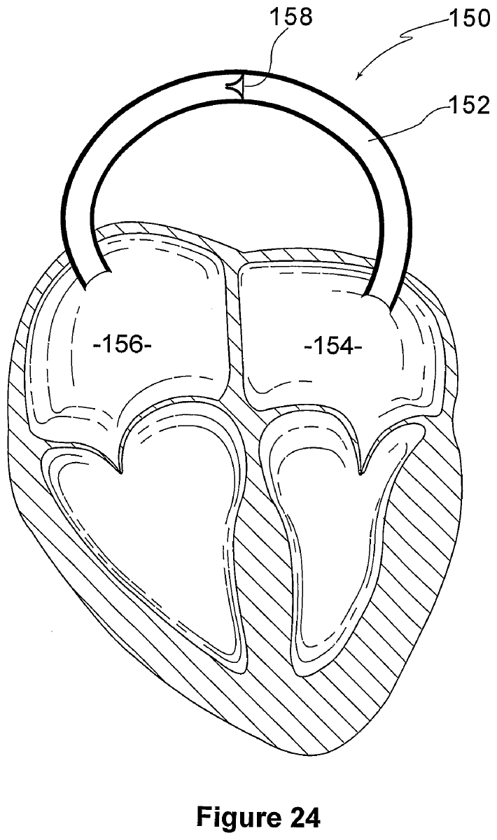

As shown in FIGS. 23a and 23b, fall deployment of the device 140 by further relative movement of the wire 144 and the head 146, relative to the catheter 142, results in the remainder of the device 140 expanding to form the second flange 14. The device 140 is initially attached to the head 146 by three pins 148, which are remotely released after the device has been deployed from the catheter 142.

In other similar embodiments (not shown) the catheter 142 has a diameter of 4-6 mm and the device 140 is initially attached to the head 146 by one or two releasable pins 148.

FIG. 20 shows a twelfth embodiment of a device 150 for treating heart failure. In this embodiment, a tube 152 of about 8 mm internal diameter provides an external fluid communication path between the heart's left and right atriums 154 and 156 respectively. A valve 158 is adapted to selectively occlude the tube 152. As with earlier embodiments, when the left atrial pressure exceeds the right atrial pressure by about 5-15 mm Hg, the valve 158 is released to open the interior of the tube 152 and allow blood flow from the left atrium 24 to the right atrium 26. In a variation of this embodiment, the valve 158 is a one way valve that prevents blood flow from the right atrium 156 to the left atrium 154.

Although the invention has been described with reference to the specific examples it will be appreciated by those skilled in the art that the invention may be embodied in many other forms.

1. (canceled)

2. A device for treating a heart condition in a patient, the device comprising: a body adapted and configured to self-expand from a radially collapsed configuration to an expanded configuration, the device in its expanded configuration comprising a first flange, a second flange, and a central portion extending between the first and second flanges, the first and second flanges each having diameters that gradually increase with distance from the central portion, respectively, the body being configured to be delivered percutaneously to a patient's heart and allowed to self-expand in an opening in a septum of the heart to dispose the first flange in the left atrium, the second flange in the right atrium and the central portion in the opening to permit blood to flow through the body from the left atrium to the right atrium.

3. The device of claim 2, further comprising a valve disposed in the body.

4. The device of claim 3, further comprising structure supporting the valve in the body.

5. The device of claim 3, wherein the valve is biased to a closed position.

6. The device of claim 5, wherein the valve is configured to open when pressure differential between the left atrium and the right atrium is 2 mm Hg or greater.

7. The device of claim 2, wherein the body comprises mesh.

8. The device of claim 7, wherein the body comprises Nitinol mesh.

9. The device of claim 2, wherein the first and second flanges are sized and configured to engage the septum at the opening when the body is expanded in the opening.

10. The device of claim 2, wherein the body is elongated in the radially collapsed configuration.
